(19) United States
(12) Patent Application Publication
(10) Pub. No.: US 2025/0283893 A1
(43) Pub. Date: Sep. 11, 2025

(54) METHOD FOR THE PROGNOSIS OF PATIENTS SUFFERING FROM ATRIAL FIBRILLATION

(71) Applicants: FUNDACIÓN INSTITUTO DE INVESTIGACIÓN SANITARIA DE SANTIAGO DE COMPOSTELA (FIDIS), Santiago de Compostela, A Coruña (ES); SERVIZO GALEGO DE SAÚDE (SERGAS), Santiago de Compostela, A Coruña (ES)

(72) Inventors: Sonia EIRAS PENAS, Santiago de Compostela, A Coruña (ES); Moisés RODRÍGUEZ-MAÑERO, Santiago de Compostela, A Coruña (ES)

(21) Appl. No.: 18/559,281

(22) PCT Filed: May 6, 2022
(86) PCT No.: PCT/EP2022/062375

§ 371 Date: Nov. 6, 2023

(30) Foreign Application Priority Data
May 7, 2021 (EP) 21382416.2

(51) Int. Cl. G01N 33/68

(52) U.S. Cl. CPC G01N 33/6893; G01N 2800/326; G01N 2800/52

(57) ABSTRACT

The present invention refers to a method for predicting the response of patients suffering from atrial fibrillation (AF) to catheter ablation treatment, or for the prognosis of patients suffering from AF.

### BACKGROUND

### Technical Field

The present invention pertains to the medical field. Particularly, the present invention refers to a method for predicting the response of patients suffering from atrial fibrillation (AF) to catheter ablation treatment, or for the prognosis of patients suffering from AF.

### Description of the Related Art

Diverse pathological processes affecting the atria contribute to the genesis and perpetuation of AF. The term ‘atrial cardiomyopathy’ has been introduced to describe changes affecting the atria with potential clinical implications and a histological/pathophysiological classification scheme has been proposed to outline the dominant underlying pathology. However, nowadays, little is known regarding the best way to characterize atrial cardiomyopathy. Tissue fibrosis can be detected and quantified as low voltage area (LVA) below specific selected thresholds at the time of the electroanatomic mapping (EAM). Although the measured voltage is influenced by several factors, atrial cardiomyopathy assessment can be inferred at the time of AF ablation. This underlying LVA is relevant first in terms of a modulator of the strategy to be implemented and second as a marker of success after AF catheter ablation procedures. It has been previously proposed that pulmonary vein isolation (PVI) only is enough if the identified LVAs does not exceed 10% of the left atrial (LA) surface area. A major limitation is that this information is not available until the procedure and other non-invasive methods for determining the extent of atrial fibrosis are limited.

Although some strategies have been previously suggested to reflect the level of atrial fibrosis, conflicting evidence exists regarding the utility of biomarkers in predicting the presence of atrial cardiomyopathy, at least determined by EAM, and their relevance in terms of recurrence after AF ablation.

So, there is an unmet medical need of finding reliable strategies focused on predicting the response of patients suffering from AF to catheter ablation treatment, or on prognosing patients suffering from AF, as an indication of the possible presence of underlying fibrotic scar and arrhythmia recurrence after catheter ablation treatment.

The present invention aims at solving this problem and a method for predicting the response of patients suffering from AF to catheter ablation treatment, or for the prognosis of AF, is herein provided.

### BRIEF SUMMARY

As explained above, the present invention refers to a method for predicting the response of patients suffering from AF to catheter ablation treatment, or for the prognosis of patients suffering from AF.

In general terms, the inventors of the present invention have tested the ability of circulating biomarkers such as Galectin-3 (Gal-3) and Fatty acid binding protein 4 (FABP4) to identify the presence of LVAs in the EAM with the intention of assessing their role in the prediction of AF recurrence after catheter ablation. Gal-3 is a protein that in humans is encoded by the LGALS3 gene. It is a member of the lectin family, of which 14 mammalian galectins have been identified. FABP4 is a master regulator of the transcription factor peroxisome proliferator-activated receptor gamma (PPARG) involved on adipocyte differentiation and, in consequence, a marker of adipose tissue inflammatory activity.

Specifically, blood samples were obtained from the left atrium and peripheral vein of consecutive patients referred for radiofrequency ablation. Bipolar voltage map was created. Areas of bipolar voltage <0.5 mV were considered as a LVA. Patients were classified into two groups LVA<10% or ≥10%. Logistic regression analysis was performed to study independent predictors for LVA. A score was achieved considering AF type (1,2,3 for paroxysmal, persistent and long-standing persistent AF), peripheral FABP4 levels (>20 ng/mL) and peripheral Gal-3 (>10 ng/mL). The Kaplan-Meier was used to describe freedom from recurrent AF for the categorical scores. Comparison of survival curves among categorical scores was performed using a log-rank test. Cox proportional hazard model was used to evaluate the effect of these categorical scores on freedom from AF recurrent.

Three hundred sixteen patients, 100 (32%) women, mean age 58±10 were included. AF was paroxysmal in 103 (33%), persistent in 131 (41%) and long-standing in 82 (27%). 73 presented LVA ≥10%. Multivariate analysis revealed gender (female), atrial or peripheral Gal-3 and FABP4 levels, LAA and AF type (long-standing persistent pattern) independent predictors for LVA ≥10%. During a mean follow-up of 655±494 days freedom of recurrence was 60%. The model including AF type+FABP4+Gal-3 stratified patients regarding freedom from recurrence, ranging from 27% (1 point) to 88% (5 points). Cox analysis determined that AF type+FABP4+Gal-3 were able to discriminate the population according the highest risk for AF recurrence after ablation (OR 5.82; CI95%1.95-17.39; p<0.001).

So, the present invention has a clear clinical relevance since the results herein provided demonstrate that peripheral FABP4 and Gal-3 levels along with AF type were able to reclassify patients according to atrial myopathy/presence of LA fibrosis along with low or high risk of AF recurrent. As a matter of fact, we have identified a subgroup of patients by combining these markers (FAB4, Gal-3, presence of LVA ≥10% and AF pattern) in whom the success rate was probably unreasonable (below 25%). Under our point of view these findings might be of interest not only for a better selection of patients referred to AF ablation but also for identifications of patients that might benefit from closer follow-up.

Consequently, it can be concluded that markers of adiposity (FABP4) and fibrosis (Gal-3) can be of particular interest in patients with persistent AF. A mechanistic “in vitro” assay showed the effect of high FABP4 levels on human atrial fibroblasts proliferation. These findings are of interest not only for a better selection of patients but also for a better understanding of the mechanism leading to AF, key to find alternative therapies.

So, the first embodiment of the present invention refers to an in vitro method for predicting the response of patients suffering from AF to catheter ablation treatment which comprises: a) Measuring the concentration level of at least [Gal-3 and FABP4] in a biological sample obtained from the patient, and b) wherein if the concentration level of at least [Gal-3 and FABP4] is statistically higher as compared with a pre-established threshold concentration level, it is an indication that the patient will not respond to catheter ablation treatment.

The second embodiment of the present invention refers to an in vitro method for the prognosis of patients suffering from AF which comprises: a) Measuring the concentration level of at least [Gal-3 and FABP4] in a biological sample obtained from the patient, and b) wherein if the concentration level of [Gal-3 and FABP4] is statistically higher as compared with a pre-established threshold concentration level, it is an indication that the patient has a bad prognosis, wherein bad prognosis indicates the presence of underlying fibrotic scar and arrhythmia recurrence after catheter ablation treatment.

In a preferred embodiment the present invention refers to an in vitro method for predicting the response of patients suffering from atrial fibrillation (AF) to catheter ablation treatment which comprises: a) Measuring the concentration level of [Gal-3 and FABP4] in a biological sample obtained from the patient, b) determining the AF type, c) processing the concentration level values of [Gal-3 and FABP4] and the AF type in order to obtain a risk score and c) wherein if the risk score obtained in the step b) is higher than a pre-established reference, is an indication that the patient will not respond to catheter ablation treatment.

In a preferred embodiment the present invention refers to an in vitro method for the prognosis of patients suffering from AF which comprises: a) Measuring the concentration level of [Gal-3 and FABP4] in a biological sample obtained from the patient, b) determining the AF type, c) processing the concentration level values of [Gal-3 and FABP4] and the AF type in order to obtain a risk score and c) wherein if the risk score obtained in the step b) is higher than a pre-established reference, is an indication that the patient has a bad prognosis, wherein bad prognosis indicates the presence of underlying fibrotic scar and arrhythmia recurrence after catheter ablation treatment.

The third embodiment of the present invention refers to the in vitro use of at least [Gal-3 and FABP4], in combination with the AF type, for predicting the response of patients suffering from AF to catheter ablation treatment, or for the prognosis of patients suffering from AF.

In a preferred embodiment, the present invention is carried out in persistent AF patients in which a catheter ablation is indicated for treatment.

The fourth embodiment refers to a kit comprising: a) Means, reagents or tools for obtaining a minimally-invasive sample from the patients (preferably plasma, blood or serum), b) Means, reagents or tools for determining the AF type and c) Means, reagents or tools for measuring the concentration level of at least [Gal-3 and FABP4].

The fifth embodiment refers to the use of the kit for predicting the response of patients suffering from AF to catheter ablation treatment, or for the prognosis of patients suffering from AF.

In a preferred embodiment, the present invention is carried out in patient suffering from AF.

In a preferred embodiment, the present invention is carried out in minimally-invasive biological samples.

In a preferred embodiment, the present invention is carried out in plasma, blood or serum.

In a preferred embodiment, the present invention refers to a method for treating AF patients by carrying out catheter ablation, which comprises a previous step of predicting the response of patients suffering from AF to catheter ablation treatment, by following the method of the invention explained above which comprises a) Measuring the concentration level of at least [Gal-3 and FABP4] in a biological sample obtained from the patient, b) determining the AF type, c) processing the concentration level values of [Gal-3 and FABP4] and the AF type in order to obtain a risk score and c) wherein the risk score obtained in the step b) is lower than a pre-established reference, is an indication that the patient will respond to catheter ablation treatment.

In a preferred embodiment, the present invention is a computer-implemented invention, wherein a processing unit (hardware) and a software are configured to:
Receive the concentration level values of the above cited biomarkers or signatures,
Receive the AF type value,
Process the concentration level values and AF type value received for finding substantial variations or deviations, and
Provide an output through a terminal display of the variation or deviation of the concentration level, wherein the variation or deviation of the concentration level indicates that the patients suffering from AF may respond or not to catheter ablation treatment.

In a preferred embodiment, the present invention refers to a method for detecting the concentration level of [Gal-3 and FABP4] in a test sample from a patient suffering from AF in order to determine whether the patient may respond or not to catheter ablation treatment, which comprises performing an immunoassay in a biological sample obtained from the patient. This embodiment may also comprise determining the AF type.

For the purpose of the present invention the following terms are defined:

The expression “minimally-invasive biological sample” refers to any sample which is taken from the body of the patient without the need of using harmful instruments, other than fine needles used for taking the blood from the patient, and consequently without being harmfully for the patient. Specifically, minimally-invasive biological sample refers in the present invention to: blood, serum, or plasma samples.

The expression “pre-established threshold concentration level,” refer to a “reference value” of the concentration level of the proteins based on mean values of the analyzed population.

The expression “risk score” refers to a risk value obtained after processing one or more values into a single value (or risk value). This risk value will be compared with a reference value to evaluate if the AF patient might respond to catheter ablation treatment.

By “comprising” it is meant including, but not limited to, whatever follows the word “comprising.” Thus, use of the term “comprising” indicates that the listed elements are required or mandatory, but that other elements are optional and may or may not be present.

By “consisting of” it is meant “including, and limited to,” whatever follows the phrase “consisting of” Thus, the phrase “consisting of” indicates that the listed elements are required or mandatory, and that no other elements may be present.

### DESCRIPTION OF THE SEVERAL VIEWS OF THE DRAWINGS

FIG. 1 shows Kaplan-Meier curves with scores estimating freedom from AF after pulmonary vein isolation: a) Score considering AF type, p<0,001; b) Score considering the combined AF type+peripheral FABP4 levels p<0,001; c) Score considering combined AF type+peripheral Gal-3 levels, p<0,001; and d) Score considering combined AF type+peripheral FABP4+peripheral Gal-3 levels, p<0,001.

FIG. 2 shows an electroanatomial map of the left atrial from two female patients with differential peripheral FABP4 and Galectin 3 levels. A) Patient 1: Woman, 68 years old. Obese (BMI 32). HBP. Persistent AF (3 years). LA area 23 cm². eGFR=86 mL/min/1.73m2. FABP=19.6 ng/mL. Galectin 3=3.35 ng/mL. B) Patient 2: Woman, 70 years old. Obese (BMI 31). HBP, DM-2 (HbA1c 7,3%). Persistent AF (9 months). LA area 24 cm². eGFR=56 mL/min/1.73 m2. FABP=55.7 ng/mL. Galectin 3=21.16 ng/mL.

FIG. 3 shows a percentage of AF recurrence on each group according to the combine score of AF pattern (+1,2,3 points) plus peripheral FABP4 (+1) and/or peripheral Galectin 3 levels >10 ng/mL (+1). Score 1 is referred to paroxysmal AF, score 2 is referred to persistent AF or paroxysmal AF with FABP4 levels >20 ng/mL or with Gal-3 levels >10 ng/mL, score 3 is referred to long-standing persistent AF or persistent AF with FABP4 levels >20 ng/mL or with Gal-3 levels >10 ng/mL, score 4 is referred to persistent AF with FABP4 levels >20 ng/mL and Gal-3 levels >10 ng/mL or long-standing persistent AF with FABP4 levels >20 ng/mL or with Gal-3 levels >10 ng/mL and score 5 is refereed to long-standing persistent AF with FABP4 levels >20 ng/mL and Gal-3 levels >10 ng/mL.

FIG. 4 shows an FABP4 effect on wound healing of human atrial fibroblasts with fetal bovine serum (FBS) determines proliferation assay. A) Optical microscopy of wound healing on human atrial fibroblasts. B) Graph bars represents mean±SD of proliferation area, in arbitrary units, with FABP4 ng/mL (1, 10 and 100 ng/mL). C) Time course of proliferation assay for 4-, 8- or 12-hours on human atrial fibroblasts.

FIG. 5 shows a survival graph representing the AF recurrence-free probability from classified patients according AF.

FIG. 6 shows a survival graph representing the AF recurrence-free probability from classified patients according Score FABGAL3 (1, 2=4, 3=5).

### DETAILED DESCRIPTION

The present invention is illustrated by means of the Examples set below without the intention of limiting its scope of protection.

### Example 1. Material and Methods

### Example 1.1. Subjects

From September 2016, through September 2020, a total of 316 consecutive patients with paroxysmal, persistent or long-standing persistent AF, classified according clinical practice guidelines, referred for pulmonary vein (PV) radiofrequency catheter ablation in a single center were included in the study. The exclusion criteria were age under 18 years, pregnancy and any latent infectious condition. All of the patients signed the informed consent. The study protocol follows the ethical guidelines of Declaration of Helsinki and approved by Ethical Committee of Clinical Research of our region according to Helsinki Declaration.

### Example 1.2. Blood Sample Collection

Before the ablation procedure (after a night of fasting), immediately after the transeptal puncture and before heparin administration, blood samples were obtained from the left atrium (LA) through the transeptal sheath. At the same time, a peripheral blood sample was obtained from an ante-cubital vein using an 18-G butterfly cannula with a two-syringe technique, discarding the first 5 mL of blood and using the second 5 mL for measures. LA and peripheral blood samples were collected in EDTA-tubes.

### Example 1.3. Plasma Measurements

After centrifuging at 1800 xg for 10 minutes, the atrial and peripheral plasma samples were stored at −80° C. until used. A magnetic Luminex multiplex test kit (R&D Systems, MN, USA) was used for analyzing FABP4 and leptin levels. The manufacturer's instructions were followed when analyzing plasma levels of FABP4 and leptin. The sensitivity for FABP4 and Leptin was 95.7 and 10.2 pg/mL respectively. Galectin-3 levels were determined using a commercially available enzyme-linked immunosorbent assay (ELISA) kit (BMS279-4; eBioscience, Vienna, Austria), according to the manufacturers' protocols. Measurements were performed in duplicate, and the results were averaged. The intra-assay and inter-assay coefficients of variation were 7.5% and 5.4%, respectively.

### Example 1.4. Ablation Procedure and Patient Follow-Up

Patients were undergoing point-by-point radiofrequency catheter ablation (with contact force sensing technology, SmartTouch, Biosense Inc.). The procedural endpoint was ipsilateral PVI. Most of the patients were discharged 24-36 hours after the procedure. Oral anticoagulation (OA) was maintained for at least 3 months (until the first medical visit). Then, OA was continued lifelong in those patients with a CHA₂DS₂-VASc score of ≥1 in men or ≥2 in women. During the blanking period (3 months), it is the standard of care in our center to continue or restart previously antiarrhythmic drug therapy (ADT). If the patient is free of recurrence after these 3 months, as evidenced by clinical evaluation and 24 h Holter recording, patients are encouraged to discontinue ADT and only restart them in case of relapse. In case of a second recurrence after ADT or electrical cardioversion if needed, and always outside the blanking period, patients are advised for a redo ablation procedure. Medical visits were systematically performed at 3, 6, 12 and 24 months after the index procedure. Each visit comprised detailed history, physical examination and 12-lead electrocardiogram (ECG).

### Example 1.5. Left Atrial Low-Voltage Areas

Bipolar voltage map was created simultaneously with LA surface reconstruction, guided by a three-dimensional EAM system (CARTO3, Biosense Webster) using a multipolar mapping catheter (PentaRay, Biosense Webster). Patients in AF rhythm at the start of the procedure systematically underwent electrical cardioversion in the electrophysiology laboratory after the transeptal puncture.

Adequate quality of the acquired voltage points was established according to CONFIDENSE module after respiratory compensation. This is a continuous mapping software with automated data acquisition when set-criteria are met, among them: 1) tissue proximity indication (proximity-based filtering of points acquired with the PentaRay); 2) wavefront annotation (an automated annotation algorithm that incorporates both unipolar and bipolar signals); 3) map consistency (system identifies outlying points when certain criteria are met; 4) position stability filter (ensures catheter position is consistent with previous location, settled in <5 mm); 5) cycle length stability (keeps data collected within a range of predefined cycle lengths, settled in 10% over the average). A minimum number of points were requested (>1000) and the density fill threshold remained constant at ≤5 mm.

Contiguous areas of bipolar voltage <0.5 mV were considered as a LVAs in sinus rhythm. Total LA surface area was defined as the LA body area without the PV antrum regions, LA appendage orifice, and mitral valve. Medians of the total LA surface area and area of each predefined region were measured offline on the three-dimensional reconstructed LA model. Median values of LVAs were set in relation to the surface area of each region and the entire LA.

### Example 1.6. Outcome'S Definition

### Presence of Low-Voltage Area on Electroanatomic Mapping

We classified patients into two groups according to the extent of the LVA<10% or ≥10% of the total LA surface.

Recurrence after AF Catheter Ablation

Recurrence was defined as electrocardiographic or Holter-documented AF, atrial flutter, or atrial tachycardia >30 seconds occurring beyond the blanking period (3 months) after the procedure.

### Example 1.7. FABP4 Effects on Human Atrial Fibroblasts (Wound Healing)

Atrial normal human cardiac fibroblasts (NHCF-A) (Lonza, Porriño, Spain) were seeded at approximately 1×10⁵ cells per well in 6-multiwell plates. Cells were starved for 8 hours. Afterwards, a would field was performed by a plastic pipette tip of 0.2 mL, with a defined gap of 1 mm in each well. Then, cells were treated with FABP4 (Cloud Clone, corp. CCC, USA) at 1, 10 or 100 ng/mL, or vehicle (3 wells per condition), with fetal bovine serum (FBS) at 10% to test migration or proliferation effects, respectively. Random fields were photographed with a microscope (Leica DMI6000B Automated/Motorized Inverted Microscope, Leica Microsystems, Wetzlar, Germany). Then area at the edge of the lesion was quantified at different times (0, 4, 8, 12, 16 and 24 h) and analyzed by ImageJ software (National Institutes of Health, Bethesda, Maryland, USA, https://imagej.nih.gov/ij/, 1997-2018). The experiment was performed by triplicate in NHCF-A between 4-6 passages

### Example 1.8. Statistical Analyses

Categorical variables were represented as percentage and continues variables as mean standard deviation (SD) or interquartile range according their normal or scatter distribution, tested by Kolmogorov-Smirnov. Population was classified according AF type and Kruskal-Wallis or chi-squared tests were used for analyzing differences among continues or categorical variables, respectively. Logistic regression analysis was performed to study independent predictors for LVA< or >than 10%. A score was achieved considering the addition of the following points: AF type (1,2,3 for paroxysmal, persistent and long-standing persistent AF, respectively), peripheral FABP4 levels >20 ng/mL (1) and peripheral Gal-3 >10 ng/mL (1). Score 1 is referred to paroxysmal AF, score 2 is referred to persistent AF or paroxysmal AF with FABP4 levels >20 ng/mL or with Gal-3 levels >10 ng/mL, score 3 is referred to long-standing persistent AF or persistent AF with FABP4 levels >20 ng/mL or with Gal-3 levels >10 ng/mL, score 4 is referred to persistent AF with FABP4 levels >20 ng/mL and Gal-3 levels >10 ng/mL or long-standing persistent AF with FABP4 levels >20 ng/mL or with Gal-3 levels >10 ng/mL and score 5 is refereed to long-standing persistent AF with FABP4 levels >20 ng/mL and Gal-3 levels >10 ng/mL. Similar score was performed with LVA>10% (1). The Kaplan-Meier was used to describe freedom from recurrent AF for the categorical scores performed with the combined of each parameter (AF type, AF type+Gal-3, AF type+FABP4, AF type+Gal-3+FABP4 with or without LVA parameter). Time-0 corresponds to the moment of PVI. Comparison of survival curves among categorical scores was performed using a log-rank test. Cox proportional hazard model was used to evaluate the effect of these categorical scores on freedom from AF recurrent. P values were penalized by Benjamini-Hochberg. All analyses were performed with Statistical Package for Social Sciences (SPSS) software (SPSS, Inc., Chicago, Ill.) and p<0.05 was considered with statistical significance.

### Example 2. Results

### Discovery Phase

### Example 2.1. Population Characteristics

Three hundred sixteen patients, 100 (32%) women, with a mean age of 58±10 years were included. AF pattern was paroxysmal in 103 (33%), persistent in 131 (41%) and long-standing persistent in 82 (27%) patients. The mean body mass index (BMI) was 29.6±4.3 kg/m². The left atrial area (LAA) average was 20±6 cm². The LVA presented a median of 1.08% (0.06-9.98). The median and interquartile range of Gal-3 levels were similar between atrial 9.2 (5.8-13.8) ng/mL and peripheral 10.1 (6.3-14.9) ng/mL. The median and interquartile range of FABP4 levels were higher in peripheral 18.9 (11.6-27.9) ng/mL than in atrial samples 15.8 (9.4-24.5) ng/mL. Most of patients were taking oral anticoagulants (direct oral anticoagulants 62% vs vitamin K antagonists 36%). About 69% of patients were receiving beta-blockers at the time of the ablation. Mean follow-up was 655±494 days and 290 patients completed more than 12 months of follow-up. Main baseline characteristics are shown on Table 1

TABLE 1

| | | | | | Percentiles | | |
|---|---|---|---|---|---|---|---|
| | % | N | Mean | SD | 25 | 50 | 75 |
| Gender (female/male) | 32/68 | 100/216 | | | | | |
| Age | | 316 | 58.4 | 10.0 | 52.0 | 59.0 | 66.8 |
| BMI (Kg/m2) | | 316 | 29.6 | 4.3 | 26.6 | 29.3 | 32.0 |
| AF type (1/2/3) | 33/41/27 | 103/129/84 | | | | | |
| LA LVAs (%) | | 293 | 9.5 | 17.8 | 0.64 | 1.08 | 9.98 |
| Years (AF) | | 310 | 3.8 | 4.6 | 1.0 | 2.0 | 5.0 |
| Redo procedure (no/yes) | 12 | 279/37 | | | | | |
| Heart Rate (bpm) | | 315 | 70.5 | 20.1 | 56.0 | 66.0 | 80.0 |
| PR (ms) | | 188 | 163.3 | 29.1 | 140 | 160.0 | 180.0 |
| QRS (ms) | | 316 | 93.5 | 15.9 | 80.0 | 90.0 | 100.0 |
| LA area (cm2) | | 304 | 20.2 | 5.6 | 16.0 | 20.0 | 24.0 |
| LVEF | | 306 | 60.2 | 9.0 | 56.0 | 61.0 | 66.0 |
| Laboratory measurements | | | | | | | |
| Hemoglobin (g/dL) | | 316 | 14.2 | 1.5 | 13.3 | 14.4 | 15.2 |
| Platelets (10\hat{ }9/L) | | 316 | 202.5 | 48.8 | 171 | 197.0 | 233.0 |
| Blood urea nitrogen (mg/dL) | | 316 | 46.2 | 15.4 | 37.0 | 45.0 | 52.0 |
| Creatinine (mg/dL) | | 316 | 1.0 | 0.3 | 0.8 | 1.0 | 1.1 |
| eGFR (mL/min/1.73 m2) | | 315 | 95.8 | 32.5 | 72.5 | 90.9 | 114.7 |
| Plasma Sodium (mEq/L) | | 316 | 140.8 | 7.5 | 140 | 141.0 | 143.0 |
| Plasma Potassium (mEq/L) | | 314 | 4.4 | 2.3 | 4.0 | 4.2 | 4.5 |
| Total cholesterol (mg/dL) | | 308 | 189.5 | 40.6 | 161 | 188.0 | 216.0 |
| LDLc (mg/dL) | | 299 | 114.6 | 32.9 | 92.0 | 115.0 | 137.0 |
| HDLc (mg/dL) | | 299 | 51.2 | 15.4 | 41.0 | 49.0 | 59.0 |
| Triglycerides (mg/dL) | | 308 | 120.8 | 58.8 | 85.0 | 108.0 | 143.5 |
| Glucose (mg/dL) | | 316 | 107.1 | 24.8 | 94.0 | 103.0 | 113.0 |
| HbA1c (g/dL) | | 220 | 5.7 | 0.6 | 5.3 | 5.6 | 5.9 |
| TSH (mU/L) | | 303 | 2.9 | 2.3 | 1.6 | 2.3 | 3.6 |
| LA Gal-3 (ng/mL) | | 229 | 10.7 | 6.6 | 5.8 | 9.2 | 13.8 |
| Peripheral Gal-3 (ng/mL) | | 229 | 11.5 | 6.9 | 6.3 | 10.1 | 14.9 |
| LA FABP4 (ng/mL) | | 314 | 20.5 | 16.7 | 9.4 | 15.8 | 24.5 |
| Peripheral FABP4 (ng/mL) | | 314 | 23.3 | 18.4 | 11.6 | 18.9 | 27.9 |
| LA Leptin (ng/mL) | | 310 | 21.0 | 30.1 | 6.2 | 11.9 | 23.5 |
| Peripheral Leptin (ng/mL) | | 311 | 23.9 | 32.2 | 7.1 | 14.7 | 27.8 |
| Disease-Risk factors | | | | | | | |
| Taquicardiomyopathy (no/yes) | 16 | 266/50 | | | | | |
| AHT (no/yes) | 44 | 178/138 | | | | | |
| T2DM (no/yes) | 12 | 278/38 | | | | | |
| Smoker (no/yes) | 29 | 223/93 | | | | | |
| COPD (no/yes) | 5 | 299/17 | | | | | |
| OSA (no/yes) | 5 | 299/17 | | | | | |
| Treatments | | | | | | | |
| Statins (no/yes) | 45 | 174/141 | | | | | |
| ACEi (no/yes) | 20 | 252/62 | | | | | |
| ARB (no/yes) | 24 | 239/76 | | | | | |
| NDHP CCB (no/yes) | 5 | 299/16 | | | | | |
| Vitamin K antagonist (no/yes) | 36 | 200/115 | | | | | |
| DOAC (no/yes) | 62 | 119/196 | | | | | |
| Class I ADT (no/yes) | 29 | 224/92 | | | | | |
| Class II ADT (no/yes) | 69 | 97/219 | | | | | |
| Class III ADT (no/yes) | 28 | 229/87 | | | | | |
| Class IV ADT (no/yes) | 7 | 293/23 | | | | | |
| Follow-up (days) | | 314 | 656 | 494 | 152 | 601 | 1096 |

BMI: Body Mass Index; AF: Atrial Fibrillation; LA: Left atrium; LVAs: Low-voltage areas; LVEF: Left Ventricular Ejection Fraction; eGFR: Estimated Glomerular Filtration Rate; Gal-3: Galecin-3; FABP4: Fatty Acid-Binding Protein 4; AHT: Arterial Hypertension; T2DM: Type 2 Diabetes Mellitus; COPD: Chronic Obstructive Pulmonary Disease; OSA: Obstructive Sleep Apnea; ACEi: Angiotensin-Converting Enzyme inhibitors; ARB: Angiotensin Receptor Blockers; NDHP CCB: Nondihydropyridine Calcium Channel Blockers; DOAC: Direct Oral Anticoagulants; ADT: Antiarrhythmic Drug Therapy.

### Example 2.2. Extent of Low-Voltage Area on EAM

Out of the 316 patients, 73 presented LVA ≥10%. This group was represented by higher percentage of patients with type 2 diabetes mellitus (T2DM) and arterial hypertension (AHT), larger LAA and older age. They presented a trend towards lower estimated glomerular filtrated rate (eGFR). See Table 2 showing clinical characteristics of patients regarding LVAs< or ≥10%.

TABLE 2

| | <10% LVAs (n = 220) | | ≥10% LVAs (n = 73) | | P value |
|---|---|---|---|---|---|
| Gender (female/male) | 66%/79% | | 34%/21% | | 0.059 |
| Age | 57 ± 10 | | 62 ± 9 | | 0.000 |
| BMI (Kg/m2) | 29 ± 4 | | 31 ± 5 | | 0.693 |
| AF type (1/2/3) | 85%/73%/66% | | 15%/27%/34% | | 0.064 |
| LA LVAs (%) | 0.4 | (0.1-2.8) | 27 | (15-42) | 0.000 |
| Years (AF) | 2 | (1-4) | 3 | (1-5) | 0.618 |
| Heart Rate (bpm) | 70 ± 20 | | 71 ± 19 | | 0.793 |
| PR (ms) | 162 ± 30 | | 164 ± 28 | | 0.852 |
| QRS (ms) | 90 | (80-100) | 95 | (80-100) | 0.501 |
| LA area (cm2) | 19 ± 5 | | 22 ± 6 | | 0.000 |
| LVEF | 61 ± 8 | | 58 ± 11 | | 0.352 |
| Laboratory measurements | | | | | |
| Hemoglobin (g/dL) | 14 ± 1 | | 14 ± 2 | | 0.488 |
| Platelets (10\hat{ }9/L) | 208 ± 50 | | 193 ± 41 | | 0.067 |
| Blood urea nitrogen (mg/dL) | 46 ± 15 | | 47 ± 18 | | 0.429 |
| Creatinine (mg/dL) | 0.9 ± 0.3 | | 1.0 ± 0.3 | | 0.747 |
| eGFR (mL/min/1.73 m2) | 100 ± 32 | | 89 ± 33 | | 0.062 |
| Plasma Sodium (mEq/L) | 141 ± 8.8 | | 141 ± 2.0 | | 0.693 |
| Plasma Potassium (mEq/L) | 4.2 | (4.0-4.5) | 4.2 | (4.1-4.4) | 0.921 |
| Total cholesterol (mg/dL) | 188 | (163-216) | 190 | (157-217) | 1.000 |
| LDLc (mg/dL) | 116 | (94-138) | 113 | (88-134) | 0.693 |
| HDLc (mg/dL) | 48 | (40-57) | 54 | (44-62) | 0.054 |
| Triglycerides (mg/dL) | 112 | (88-147) | 97 | (79-122) | 0.064 |
| Glucose (mg/dL) | 103 | (94-113) | 104 | (93-115) | 0.693 |
| HbA1c (g/dL) | 5.7 ± 0.5 | | 5.9 ± 0.7 | | 0.316 |
| TSH (mU/L) | 2.38 | (1.5-3.7) | 2.20 | (1.54-3.70) | 0.693 |
| LA Gal-3 (ng/mL) | 8.9 | (5.7-12.7) | 12 | (4.7-15) | 0.300 |
| Peripheral Gal-3 (ng/mL) | 9.4 | (6.5-14) | 13 | (5.7-17) | 0.316 |
| LA FABP4 (ng/mL) | 15.3 | (9.17-23) | 19 | (11-30) | 0.066 |
| Peripheral FABP4 (ng/mL) | 18.4 | (11-27) | 22 | (12-38) | 0.212 |
| LA Leptin (ng/mL) | 11.7 | (6-23) | 13 | (7.4-26) | 0.584 |
| Peripheral Leptin (ng/mL) | 14.2 | (7-27) | 16 | (7.6-33) | 0.676 |

| | | | |
|---|---|---|---|
| Disease and risk factors | | | |
| AHT (no/yes) | 83%/17% | 65%/35% | 0.000 |
| T2DM (no/yes) | 78%/22% | 54%/46% | 0.017 |
| Smoker (no/yes) | 75%/26% | 76%/24% | 0.879 |
| COPD (no/yes) | 75%/25% | 71%/29% | 0.852 |
| OSA (no/yes) | 75%/25% | 71%/29% | 0.852 |
| Treatments | | | |
| Statins (no/yes) | 76%/24% | 74%/26% | 0.325 |
| ACEi (no/yes) | 77%/23% | 68%/32% | 0.064 |
| ARB (no/yes) | 79%/21% | 64%/36% | 0.693 |
| NDHP CCB (no/yes) | 76%/25% | 67%/33% | 0.292 |
| Vitamin K antagonist (no/yes) | 78%/22% | 69%/31% | 0.292 |
| DOAC (no/yes) | 70%/30% | 78%/22% | 0.852 |
| Class I ADT (no/yes) | 74%/26% | 77%/23% | 1.000 |
| Class II ADT (no/yes) | 75%/25% | 75%/25% | 0.793 |
| Class III ADT (no/yes) | 74%/26% | 77%/23% | 0.352 |
| Class IV ADT (no/yes) | 76%/24% | 62%/38% | 0.135 |
| Event (no/yes) | 79%/21% | 69%/31% | 0.693 |
| Time of event (days) | 698 ± 491 | 537 ± 500 | 0.227 |

BMI: Body Mass Index; AF: Atrial Fibrillation; LA: Left atrium; LVAs: Low-voltage areas; LVEF: Left Ventricular Ejection Fraction; eGFR: Estimated Glomerular Filtration Rate; Gal-3: Galecin-3; FABP4: Fatty Acid-Binding Protein 4; AHT: Arterial Hypertension; T2DM: Type 2 Diabetes Mellitus; COPD: Chronic Obstructive Pulmonary Disease; OSA: Obstructive Sleep Apnea; ACEi: Angiotensin-Converting Enzyme inhibitors; ARB: Angiotensin Receptor Blockers; NDHP CCB: Nondihydropyridine Calcium Channel Blockers; DOAC: Direct Oral Anticoagulants; ADT: Antiarrhythmic Drug Therapy.

Multivariate analysis revealed gender (female), atrial or peripheral Gal-3 and FABP4 levels, LAA and AF type (long-standing persistent pattern) independent predictors for LVA≥10%. Please refer to Table 3a and 3b showing the logistic regression analysis: dependent variable LVAs< or ≥10%.

TABLE 3

| | |
|---|---|
| OR (95% CI) | Sig. |

a) LA Gal-3 and FABP4 levels

| | | |
|---|---|---|
| Age | 1.024 (0.975-1.084) | 0.383 |
| Gender (male) | 0.400 (0.174-0.917) | 0.031 |
| LA Gal-3 | 1.099 (1.042-1.160) | 0.001 |
| LA FABP4 | 1.020 (1.001-1.040) | 0.039 |
| T2DM | 1.870 (0.615-5.680) | 0.270 |
| eGFR | 0.998 (0.982-1.014) | 0.998 |
| AF type (long-standing | 3.190 (1.200-8.490) | 0.020 |
| persistent) | | |
| AHT | 1.260 (0.570-2.80) | 0.567 |
| BMI | 0.944 (0.860-1.035) | 0.219 |
| LA area | 1.137 (1.057-1.224) | 0.001 |

b) Peripheral Gal-3 and FABP4 levels

| | | |
|---|---|---|
| Age | 1.027 (0.975-1.082) | 0.311 |
| Gender (male) | 0.425 (0.185-0.973) | 0.043 |
| Peripheral Gal-3 | 1.094 (1.037-1.153) | 0.001 |
| Peripheral FABP4 | 1.018 (1.000-1.036) | 0.047 |
| T2DM | 1.787 (0.584-5.472) | 0.309 |
| eGFR | 0.998 (0.982-1.014) | 0.808 |
| AF type (long-standing | 3.142 (1.182-8.352) | 0.022 |
| persistent) | | |
| AHT | 1.334 (0.606-2.937) | 0.474 |
| BMI | 0.947 (0.865-1.037) | 0.247 |
| LA area | 1.143 (1.061-1.232) | 0.000 |

LA: Left atrium; Gal-3: Galecin-3; FABP4: Fatty Acid-Binding Protein 4; eGFR: Estimated Glomerular Filtration Rate; AF: Atrial Fibrillation; AHT: Arterial Hypertension; BMI: Body Mass Index

### Example 2.3. Recurrence after AF Catheter Ablation

Our population presented 60% freedom of recurrence during follow-up. Patients with long-standing persistent AF pattern had recurrence in a shortened period of time as compared to patients with persistent and paroxysmal pattern (496±442 vs 655±519 vs 782±467 days; p<0.001).

A score was made by assigning 1, 2, 3 points if the AF pattern was paroxysmal, persistent or long-standing persistent, respectively. Then, 1 point was added to AF type if peripheral plasma FABP4 levels were greater than 20 ng/mL and another point in case of Gal-3 greater than 10 ng/mL. This led to a final score of up to 5 points in which a punctuation of 1 was assigned to paroxysmal AF and 5 points was assigned to long-standing persistent AF with peripheral levels of FAPB4 and Gal-3 greater than 20 ng/ml and 10 ng/ml, respectively.

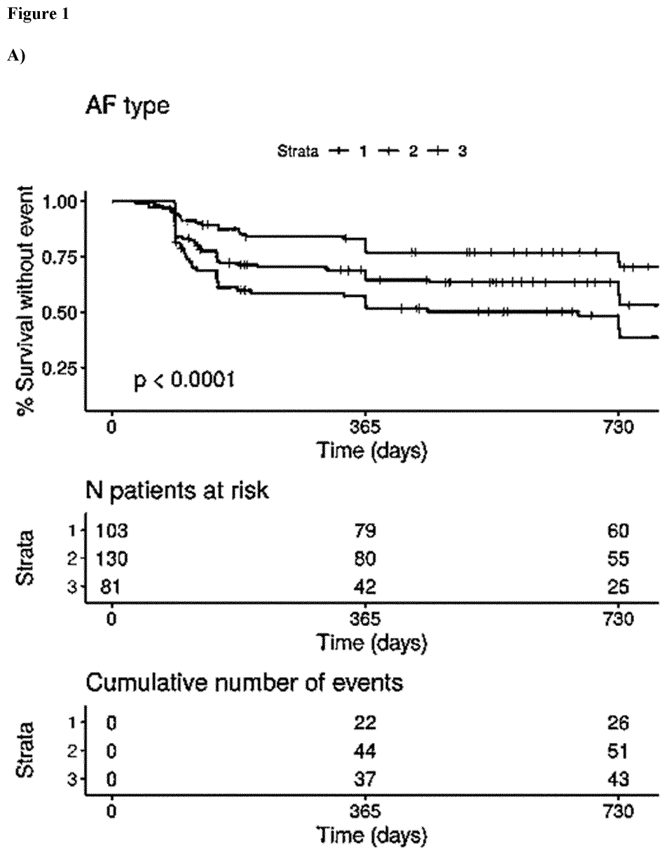

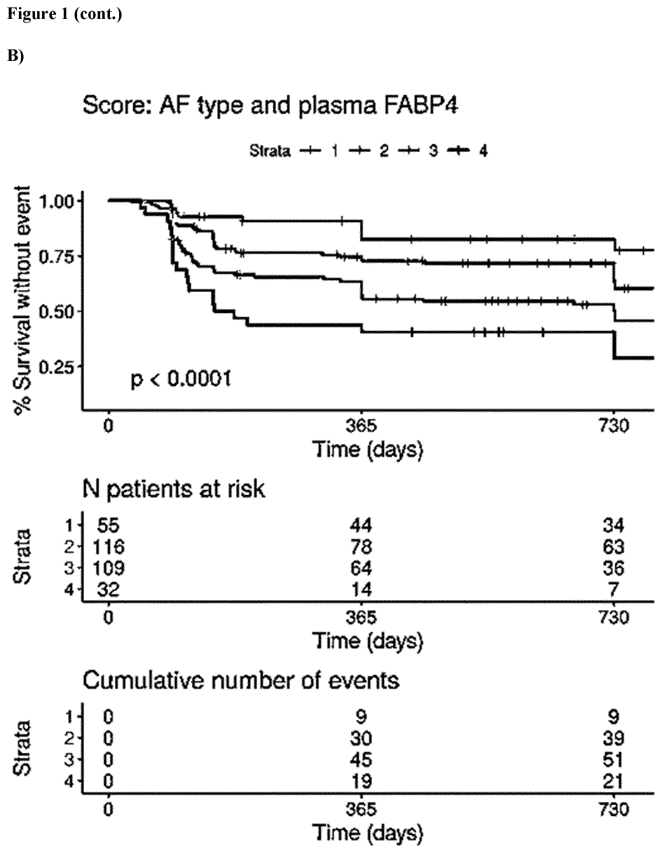

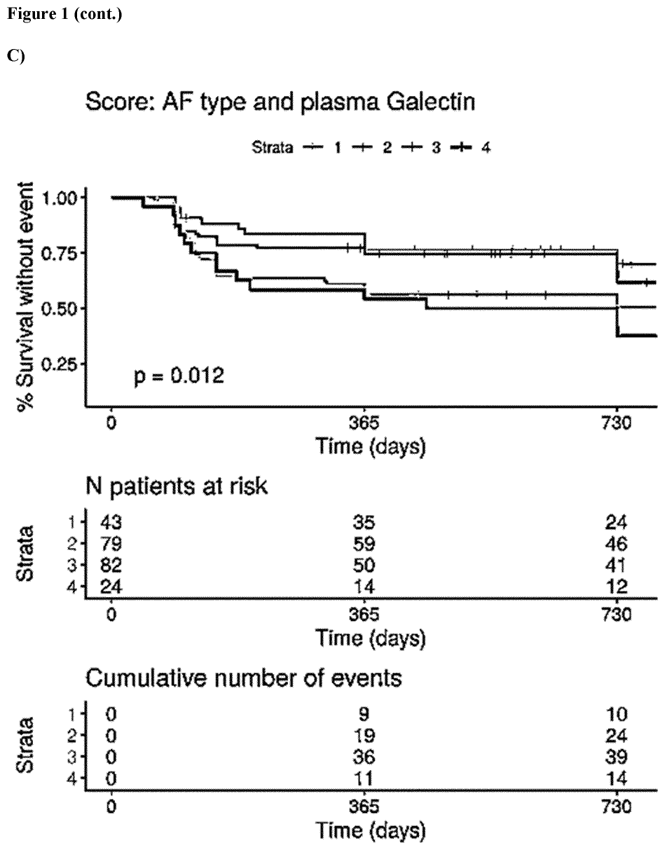

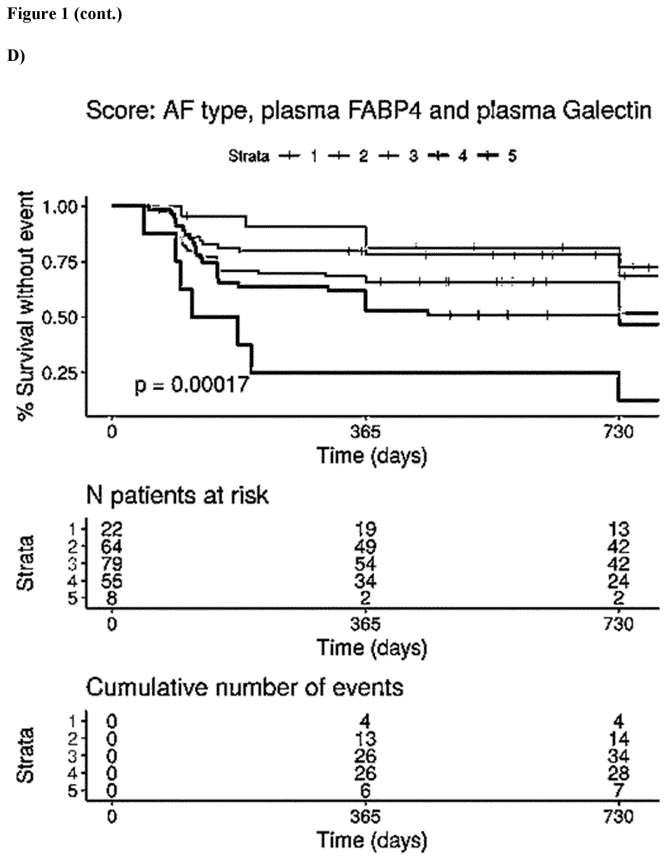

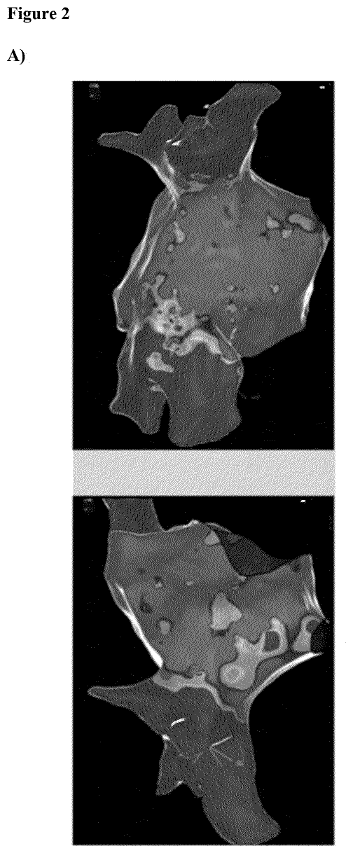

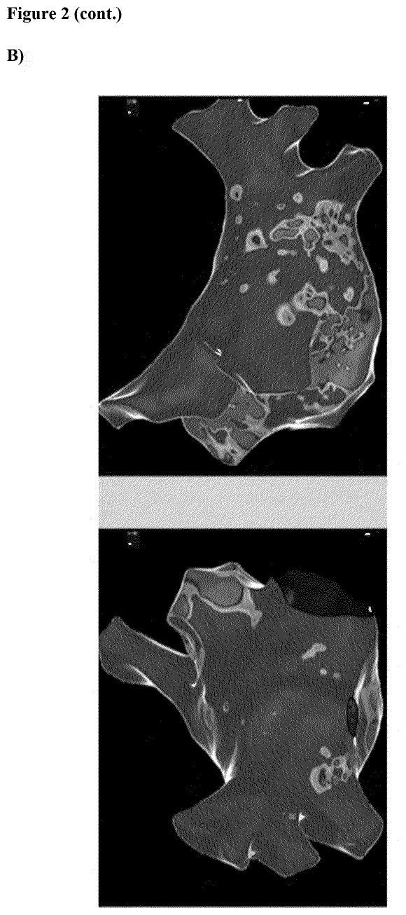

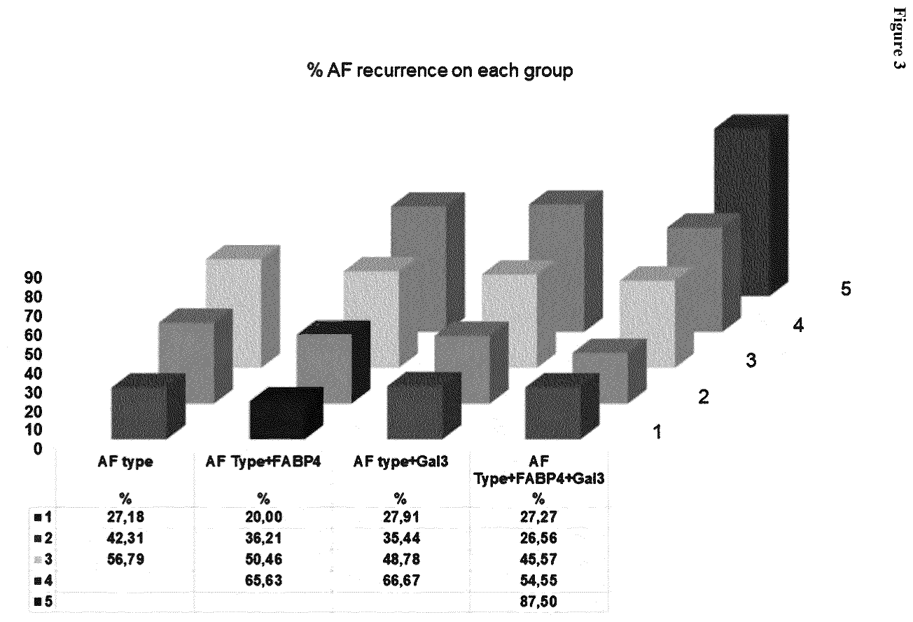

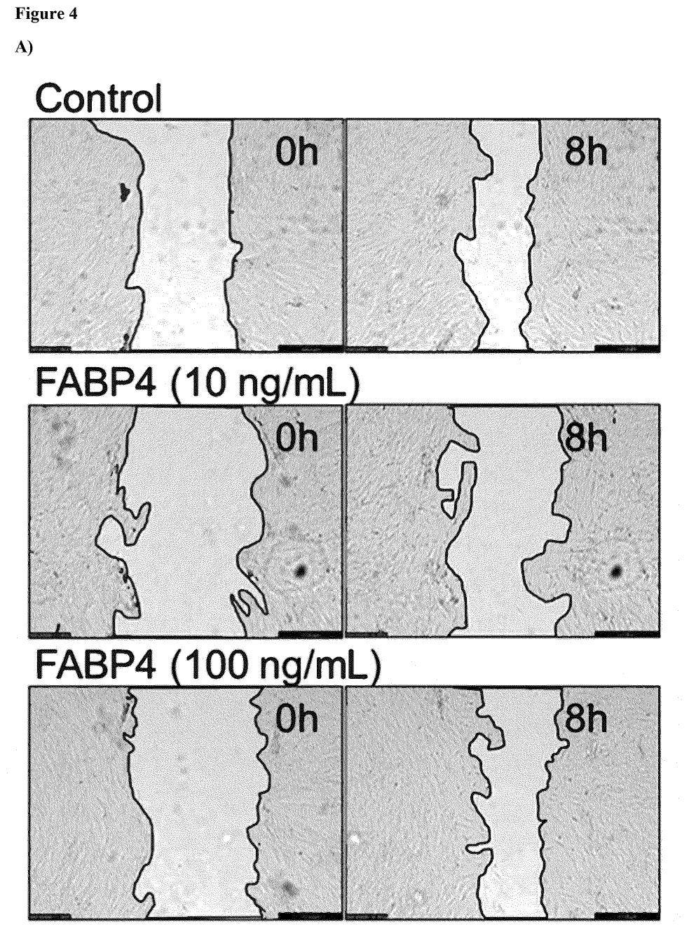

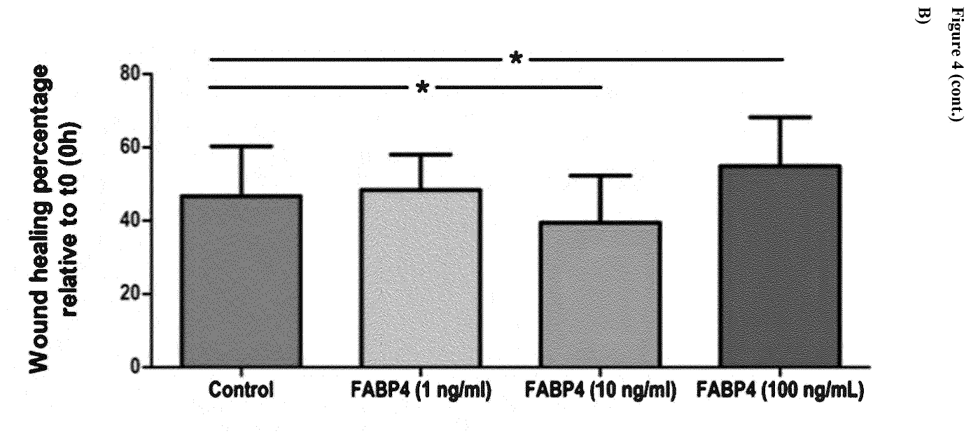

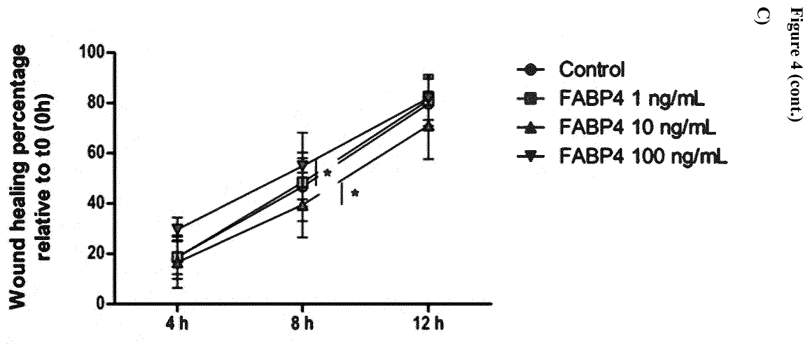

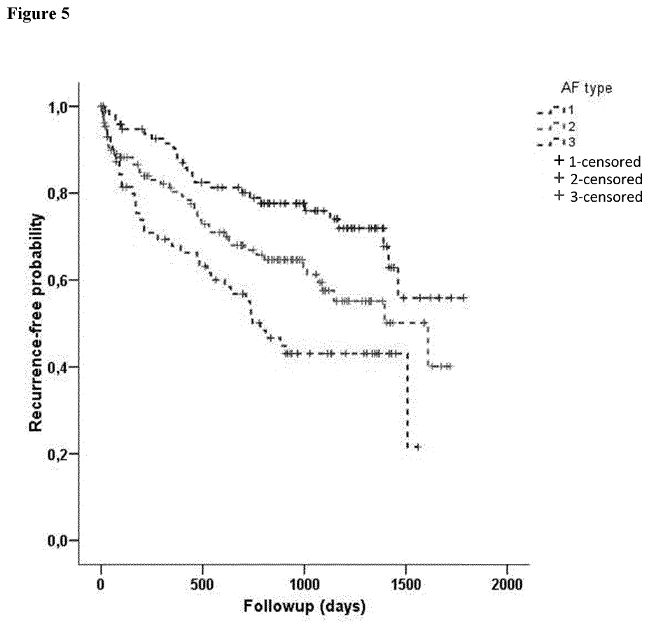

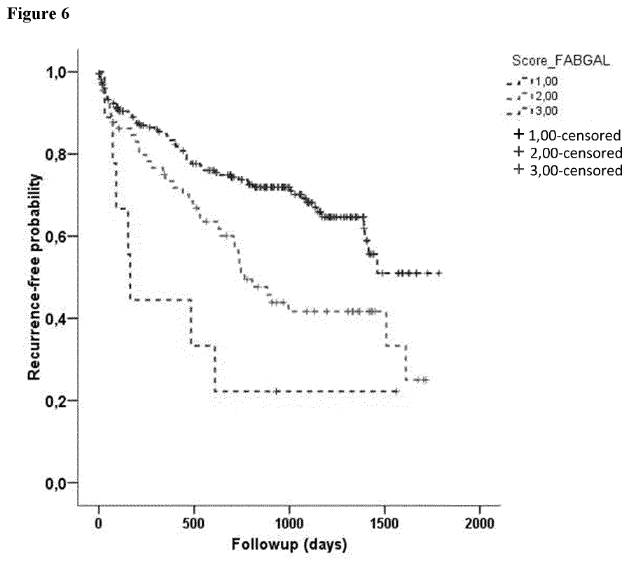

The Kaplan Meier showed that AF type, AF type+FABP4, AF type+Gal-3 and AF type+FABP4+Gal-3 stratified patients regarding freedom from recurrence (FIG. 1). As shown in the Kaplan Meir curves, the present score reclassified patients in a more efficient manner than the conventional AF classification; the AF recurrent was detected in 27% of patients classified as score 1 and in 88% of patients classified as score 5 (FIG. 2), specifically, the Cox regression analysis determined that AF type combined with FABP4 and Gal-3 were able to discriminate the population according the highest risk for AF recurrence after ablation 5.82 (1.95-17.39), p<0.001. The model with AF type alone showed a higher risk for recurrent AF in those patients with long-persistent AF 2.80 (1.75-4.49), the model with AF type and added FABP4 levels >20 ng/mL showed a higher risk for AF recurrent in those patients with long-standing persistent AF with higher FABP4 levels than 20 ng/mL 5.25 (2.52-10.91) and the model with AF type added Gal-3 showed a higher risk for AF recurrent in those patients with long-standing persistent AF with higher Gal-3 levels than 10 ng/mL 2.81(1.33-5.94). Same procedure was performed with LVA ≥10% and FABP4 or Gal-3 and AF type. An additional point was added in patients with LVA ≥10% (Supplementary FIG. 1). The Log-rank showed statistical significance on every stratification groups. We have included in a Cox regression multivariate analysis the following variables (age, AHT, gender, LA area, T2DM, eGFR, AF type+Gal-3+FABP4). It showed that the combined indicator (AF type+Gal-3+FABP4) had the highest odds ratio (6,200 (1,847-20,817)) with statistical significance (Table 4) for AF recurrence.

TABLE 4

| | OR (95% CI) | Sig. |
|---|---|---|
| Age | 1.007 (0.979-1.035) | 0.632 |
| Gender (male) | 0.458 (0.287-0.730) | 0.001 |
| LA area | 1.064 (1.026-1.103) | 0.001 |
| AHT | 1.326 (0.854-2.059) | 0.208 |
| T2DM | 0.723 (0.343-1.525) | 0.394 |
| eGFR | 1.004 (0.996-1.012) | 0.359 |
| AF type_Gal3_FABP4 | | |
| AF type_Gal3_FABP4 (2) | 0.954 (0.360-2.527) | 0.924 |
| AF type_Gal3_FABP4 (3) | 2.211 (0.890-5.492) | 0.087 |
| AF type_Gal3_FABP4 (4) | 2.738 (1.093-6.860) | 0.032 |
| AF type_Gal3_FABP4 (5) | 6.200 (1.847-20.817) | 0.003 |

LA: Left atrium; AHT: Arterial Hypertension; T2DM: Type 2 Diabetes Mellitus; eGFR: Estimated Glomerular Filtration Rate; AF: Atrial Fibrillation; Gal-3: Galecin-3; FABP4: Fatty Acid-Binding Protein 4.

### Example 2.4. FABP4 Effects on Human Atrial Fibroblasts

The “in vitro” assay showed a reduction of fibroblasts proliferation rate after treatment with FABP4 at 10 ng/mL (46.69±13.63 arbitrary units (a.u.) control vs 39.43±12.88 a.u. FABP4 10 ng/mL, p=0.0164). However, the highest FABP4 concentration (100 ng/mL) enhanced the proliferation rate of human atrial fibroblasts (46.69±13.63 a.u. control vs 54.91±13.31 a.u. FABP4 at 100 ng/mL, p=0.0266) after 8 hours of treatment (FIG. 3).

### Validation Phase

### Example 2.5. Validation Phase

We have included 303 patients with atrial fibrillation (AF) undergoing pulmonary vein isolation (PVI) (from 2016 until 2021) and Galectin-3 and fatty acid binding protein 4 (FABP4) analyzed on peripheral vein plasma. The main clinical characteristics of patients are included in Table 5.

TABLE 5

| | | | | | Percentiles | | |
|---|---|---|---|---|---|---|---|
| | % | N | Mean | SD | 25 | 50 | 75 |
| Gender (female/male) | 32/68 | 98/205 | | | | | |
| Age (years) | | 303 | 58.2 | 10.0 | 52.0 | 59.0 | 66.0 |
| BMI (Kg/m2) | | 303 | 29.7 | 4.8 | 26.7 | 29.4 | 32.0 |
| AF type (1/2/3) | 33/44/23 | 100/132/71 | | | | | |
| LA LVAs (%) | | 272 | 9.6 | 17.8 | 0.06 | 1.44 | 9.98 |
| Redo procedure (no/yes) | 11 | 268/35 | | | | | |
| Heart Rate (bpm) | | 278 | 70.0 | 21.5 | 56.0 | 65.0 | 80.0 |
| PR (ms) | | 165 | 161.4 | 31.5 | 140 | 160.0 | 180.0 |
| QRS (ms) | | 275 | 92.7 | 15.4 | 80.0 | 90.0 | 100.0 |
| LA area (cm2) | | 281 | 20.4 | 6.0 | 15.0 | 20.0 | 24.0 |
| LVEF | | 291 | 60.6 | 9.0 | 56.0 | 61.0 | 66.0 |
| Laboratory | | | | | | | |
| Hemoglobin (g/dL) | | 301 | 14.3 | 1.4 | 13.4 | 14.4 | 15.3 |
| Platelets (10\hat{ }9/L) | | 303 | 201.9 | 50.5 | 170 | 200.0 | 235.0 |
| Blood urea nitrogen (mg/dL) | | 303 | 47.0 | 15.7 | 38.0 | 45.0 | 53.0 |
| Creatinine (mg/dL) | | 303 | 1.3 | 3.4 | 0.8 | 1.0 | 1.1 |
| Plasma Na (mEq/L) | | 303 | 141.0 | 7.6 | 140 | 141.0 | 143.0 |
| Plasma K(mEq/L) | | 303 | 4.5 | 3.63 | 4.0 | 4.2 | 4.5 |
| Total cholesterol (mg/dL) | | 296 | 189.0 | 39.4 | 161 | 188.0 | 215.0 |
| LDLc (mg/dL) | | 286 | 115.0 | 32.1 | 92.0 | 115.0 | 137.0 |
| HDLc (mg/dL) | | 284 | 51.3 | 15.7 | 41.0 | 50.0 | 59.0 |
| Triglycerides (mg/dL) | | 296 | 118.0 | 57.5 | 84.0 | 106.0 | 139.0 |
| Glucose (mg/dL) | | 303 | 106.7 | 24.0 | 93.0 | 103.0 | 113.0 |
| HbA1c (g/dL) | | 199 | 5.7 | 0.5 | 5.4 | 5.6 | 5.9 |
| TSH (mU/L) | | 303 | 2.9 | 3.5 | 1.6 | 2.3 | 3.6 |
| LA Gal-3 (ng/mL) | | 272 | 10.1 | 6.3 | 5.8 | 8.6 | 12.9 |
| Peripheral Gal-3 (ng/mL) | | 303 | 10.4 | 6.8 | 5.7 | 8.5 | 13.7 |
| LA FABP4 (ng/mL) | | 274 | 20.5 | 16.7 | 9.6 | 15.9 | 24.7 |
| Peripheral FABP4 (ng/mL) | | 303 | 24.1 | 18.2 | 12.4 | 19.6 | 29.2 |
| Disease-Risk factors | | | | | | | |
| Taquicardiomyopathy (no/yes) | 82/16 | 251/48 | | | | | |
| AHT (no/yes) | 54/46 | 165/138 | | | | | |
| T2DM (no/yes) | 88/12 | 265/38 | | | | | |
| Smoker (no/yes) | 70/30 | 216/87 | | | | | |
| COPD (no/yes) | 93/7 | 282/19 | | | | | |
| OSA (no/yes) | 94/6 | 284/18 | | | | | |
| Kidney failure (no/yes) | 93/7 | 283/20 | | | | | |
| Treatments | | | | | | | |
| Statins (no/yes) | 56/44 | 171/132 | | | | | |
| ACEi (no/yes) | 80/20 | 244/59 | | | | | |
| ARB (no/yes) | 77/23 | 233/70 | | | | | |
| Vitamin K antagonist (no/yes) | 65/35 | 196/107 | | | | | |
| DOAC (no/yes) | 36/64 | 110/193 | | | | | |
| Class I ADT (no/yes) | 69/31 | 208/95 | | | | | |
| Class II ADT (no/yes) | 31/69 | 92/211 | | | | | |
| Class III ADT (no/yes) | 71/29 | 216/87 | | | | | |
| Class IV ADT (no/yes) | 93/7 | 281/22 | | | | | |

The follow-up of patients was (1-1785 days) with a median (interquartile range) 781(228-1154). The recurrence was detected in 36% of patients. The AF type and left atrial size were the main clinical characteristic associated with this event. Thus, the recurrence was represented by 260%, 360% or 5300 in paroxysmal AF, persistent AF or long-persistent AF, respectively. After selecting those patients with at least 365 days of follow-up (n=212), this variable did not define properly the group of patients with AF recurrence. We performed a univariate Cox regression analysis to determine the main predictors for AF recurrence of all included patients (n=303). The results are showing in Table 6.

TABLE 6

| | OR (95% CI) | Sig. |
|---|---|---|
| Age | 0.996 (0.978-1.014) | 0.632 |
| BMI | 0.951 (0.909-0.995) | 0.030 |
| Gender (male) | 0.756 (0.516-1.108) | 0.151 |
| Peripheral Gal-3 | 0.987 (0.959-1.016) | 0.379 |
| Peripheral Gal-3 (≥10 ng/mL = 1) | 0.945 (0.646-1.382) | 0.772 |
| Peripheral FABP4 | 1.002 (0.991-1.012) | 0.764 |
| Peripheral FABP4(≥20 ng/mL = 1) | 1.243 (0.856-1.805) | 0.253 |
| T2DM | 1.787 (0.584-5.472) | 0.309 |
| eGFR | 0.998 (0.982-1.014) | 0.808 |
| AF type (persistent) | 1.709 (1.058-2.763) | 0.029 |
| AF type (long-standing persistent) | 2.672 (1.619-4.409) | 0.000 |
| AHT | 0.997 (0.684-1.451) | 0.986 |
| Triglycerides | 0.996 (0.992-1.000) | 0.052 |
| Left atrial size | 1.054 (1.022-1.087) | 0.001 |

We performed several scores considering both variables (FABP4 or Galectin-3) and AF type:
a) FAB4 (Score=1,2,3,4) based on cut-off values of FABP4 levels (median) and AF type. The score points were AF type (1,2,3 according paroxysmal, persistent or long-persistent) and FABP4 levels ≥20 ng/mL=1.
b) FAB3 (Score 1=Score 1, 2 of FAB4, Score 3=Score 3 of FAB4, Score 4=Score 4 of FAB4)
c) FAGAL4 (Score=1,2,3,4) based on cut-off values of Galectin-3 levels (median) and AF type. The score points were AF type (1,2,3 according paroxysmal, persistent or long-persistent), Galectin-3 levels ≥10 ng/mL=1.
d) FABGAL5 (Score=1,2,3,4,5) based on cut-off values of FABP4, Galectin-3 levels (median) and AF type. The score points were AF type (1,2,3 according paroxysmal, persistent or long-persistent), FABP4 levels ≥20 ng/mL=1 and Galectin-3 levels ≥10 ng/mL=1.
e) FABGAL3 (Score 1=Score 1, 2, 3 of FABGAL5, Score 4=Score 4 of FABGAL5, Score 5=Score 5 of FABGAL5)

The univariate Cox regression analysis determined their predictive value for AF recurrence after catheter ablation (see Table 7).

TABLE 7

| | OR (95% CI) | Sig. |
|---|---|---|
| Score FAB4 | | |
| Score (2) | 1.081(0.582-2.008) | 0.805 |
| Score (3) | 1.864(1.038-3.348) | 0.037 |
| Score (4) | 3.334 (1.687-6.590) | 0.001 |
| Score FAB3 | | |
| Score (1 = Score 1 or 2) | | |
| Score (3) | 1.739 (1.155-2.621) | 0.008 |
| Score (4) | 2.918 (1.687-5.046) | 0.000 |
| Score FAGAL4 | | |
| Score (2) | 1.032 (0.581-1.833) | 0.914 |
| Score (3) | 1.614 (0.929-2.804) | 0.090 |
| Score (4) | 2.433 (1.264-4.680) | 0.008 |
| Score FABGAL5 | | |
| Score (2) | 0.824 (0.395-1.718) | 0.605 |
| Score (3) | 0.995 (0.488-2.804) | 0.090 |
| Score (4) | 1.775 (0.882-3.570) | 0.108 |
| Score (5) | 1.327 (3.496-9.210) | 0.011 |
| Score FABGAL3 | | |
| Score (1 = Score 1, 2 or 3) | | |
| Score (4) | 1.920 (1.284-2.871) | 0.001 |
| Score (5) | 3.779 (1.728-8.265) | 0.001 |

Afterwards, we performed a multivariate Cox regression analysis with the scores, AF type and left atrial size for identifying the best predictor for AF recurrence. The results showed that Score FABGAL3 and left atrial size were included in the best predictive model (see Table 8).

TABLE 8

| Score FABGAL3 | | |
|---|---|---|
| Score (1 = Score 1, 2 or 3) | OR (95% CI) | Sig. |
| Score (4) | 1.994 (1.328-2.994) | 0.001 |
| Score (5) | 3.521 (1.605-7.725) | 0.002 |
| Left atrial size | 1.054 (1.022-1.088) | 0.001 |

Our results have demonstrated that the score based on AF type, FABP4 and Galectin-3 improved the identification of patients with AF recurrent after catheter ablation. We described the patient's percentage on each score:

### % Recurrence for AF Type (Score 1,2,3):

Total: 36.75%
Score=1: 26%
Score=2: 35.60%
Score=3: 54.29%

### % Recurrence for Score FAB3:

Total: 36.75%
Score=1: 28.40%
Score=3: 41.07%
Score=4: 67.86%

### % Recurrence for FABGAL3

Total: 36.75%
Score=1:28.83%
Score=4:52.70%
Score=5: 76.92%

### % Recurrence for FABGAL5

Total: 36.75%
Score=1: 36.84%
Score=2: 30.11%
Score=3: 26.21%
Score=4: 52.70%
Score=5: 76.92%

A higher percentage of patients was identified in the most recurrent group if the score included AF type and both markers (FABP4 and Galectin-3):
A) After selecting only patients with non-paroxysmal AF (persistent and long-persistent AF), which is the clinical variable associated with AF recurrence after catheter ablation, we observed that the univariate Cox regression analysis determined the following predictors (see Table 9).

TABLE 9

| | OR (95% CI) | Sig. |
|---|---|---|
| Age | 1.007 (0.986-1.028) | 0.530 |
| BMI | 0.956 (0.908-1.007) | 0.089 |
| Gender (male) | 0.561 (0.360-0.873) | 0.011 |
| Peripheral FABP4 | 1.006 (0.995-1.018) | 0.275 |
| Peripheral Gal-3 | 1.006 (0.975-1.038) | 0.723 |
| T2DM | 0.649 (0.283-1.488) | 0.307 |
| eGFR | 0.997 (0.991-1.004) | 0.423 |
| AHT | 1.129 (0.736-1.731) | 0.580 |
| Triglycerides | 0.995 (0.991-1.000) | 0.041 |
| Left atrial size | 1.038 (1.000-1.077) | 0.050 |
| Peripheral FABP4 (≥20 ng/mL = 1) | 1.451 (0.944-2.229) | 0.089 |
| Peripheral Gal-3 (≥10 ng/mL = 1) | 1.097 (0.710-1.689) | 0.675 |

After splitting the population with respect sex and including in a multivariate Cox regression analysis the following variables (body mass index, left atrial size, triglycerides and Peripheral FABP4 (≥20 ng/mL=1) or Galectin-3 (≥10 ng/mL=1), we found that the best predictor for AF recurrence was left atrial size and Peripheral FABP4 (≥20 ng/mL=1) in male, as it is shown below, but it did not reach the statistical significance in female (see Table 10).

TABLE 10

| | OR (95% CI) | Sig. |
|---|---|---|
| BMI | 0.933 (0.870-1.001) | 0.053 |
| Triglycerides | 0.997 (0.993-1.002) | 0.248 |
| Left atrial size | 1.061 (1.009-1.116) | 0.021 |
| Peripheral FABP4 (≥20 ng/mL = 1) | 1.815 (1.018-3.234) | 0.043 |

After considering the Score FAB3 in the Cox regression model, we observed a better predictive value (see Table 11).

TABLE 11

| | OR (95% CI) | Sig. |
|---|---|---|
| BMI | 0.929(0.865-0.998) | 0.044 |
| Triglycerides | 0.998 (0.993-1.002) | 0.299 |
| Left atrial size | 1.055 (1.002-1.110) | 0.042 |
| Score FAB3 | | 0.025 |
| Score FAB3 (Score = 3) | 1.799 (0.929-3.485) | 0.082 |
| Score FAB3 (Score = 4) | 3.374 (1.382-8.234) | 0.008 |

After considering the Score FABGAL3 in the regression model, we observed a predictive value in women and in men:
1. In women (Table 12)

TABLE 12

| | OR (95% CI) | Sig. |
|---|---|---|
| BMI | 0.913 (0.819-1.018) | 0.024 |
| Triglycerides | 0.986 (0.972-1.001) | 0.059 |
| Left atrial size | 1.073 (1.007-1.143) | 0.029 |
| FABGAL3 (Score = 4) | 2.289 (1.006-5.208) | 0.048 |
| FABGAL3 (Score = 5) | 2.640 (0.674-10.340) | 0.163 |

2. In men (Table 13)

TABLE 13

| | OR (95% CI) | Sig. |
|---|---|---|
| BMI | 0.917 (0.851-0.988) | 0.023 |
| Triglycerides | 0.998 (0.993-1.002) | 0.297 |
| Left atrial size | 1.074 (1.021-1.129) | 0.006 |
| FABGAL3 (Score = 4) | 1.266 (2.329-4.284) | 0.007 |
| FABGAL3 (Score = 5) | 4.743 (1.575-14.285) | 0.006 |

We described the patient's percentage on each score for women (n=54) and recurrence. Out of 54, 40 women were grouped in FABP4≥20 ng/mL=1.

% Recurrence for FABP4≥20 ng/mL=1: 55%

### % Recurrence for Score FAB3 (Score 1,3,4):

Total: 57.4%
Score=1 (n=8): 75%
Score=3 (n=32): 46.9%
Score=4 (n=14): 71.4%

### % Recurrence for Score FABGAL3:

Total: 57.4%
Score=1 (n=27): 48%
Score=4 (n=23): 65%
Score=5 (n=4): 75%

We described the patient's percentage on each score for men (n=148). Out of 148, 57 men were grouped in FABP4≥20 ng/mL=1.

% Recurrence for FABP4≥20 ng/mL=1: 42.1%

### % Recurrence for Score FAB3 (Score 1,3,4):

Total: 36.5%
Score=1 (n=55): 25.5%
Score=3 (n=78): 39.7%
Score=4 (n=15): 60%

### % Recurrence for Score FABGAL3:

Total: 36.5%
Score=1 (n=100): 28%
Score=4 (n=43): 51.2%
Score=5 (n=5): 80%

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An In vitro method for predicting the response of patients suffering from atrial fibrillation (AF) to catheter ablation treatment which comprises: a) measuring the concentration level of at least [Gal-3 and FABP4] in a biological sample obtained from the patient, b) determining the AF type, c) processing the concentration level values of [Gal-3 and FABP4] and the AF type in order to obtain a risk score and c) wherein if the risk score obtained in the step b) is higher than a pre-established reference, is an indication that the patient will not respond to catheter ablation treatment.

2. An In vitro method for the prognosis of patients suffering from atrial fibrillation (AF) which comprises: a) measuring the concentration level of [Gal-3 and FABP4] in a biological sample obtained from the patient, b) determining the AF type, c) processing the concentration level values of [Gal-3 and FABP4] and the AF type in order to obtain a risk score and c) wherein if the risk score obtained in the step b) is higher than a pre-established reference, is an indication that the patient has a bad prognosis, wherein bad prognosis indicates the presence of underlying fibrotic scar and arrhythmia recurrence after catheter ablation treatment.

3. The In vitro method; according to claim 1, wherein the method is carried out in persistent AF patients in which a catheter ablation is indicated for treatment.

4. (canceled)

5. The In vitro method; according to claim 1, wherein the biological sample is a minimally-invasive biological sample.

6. The In vitro method; according to claim 1, wherein the biological sample is plasma, blood or serum.

7. (canceled)

8. The In vitro method; according to claim 1, wherein the patient has persistent AF in which a catheter ablation is indicated for treatment.

9. The In vitro method according to claim 8, wherein the biological sample is a minimally-invasive biological sample obtained from the patient.

10. The In vitro method according to claim 9, wherein the biological sample is plasma, blood or serum obtained from the patient.

11. A kit comprising:
a. means, reagents or tools for obtaining a minimally-invasive sample from the patients,
b. means, reagents or tools for measuring the concentration level of at least [Gal-3 and FABP4], and
c. means, reagents or tools for determining the AF type.

12. The kit, according to claim 11, comprising:
a. means, reagents or tools for obtaining plasma, blood or serum from the patients,
b. means, reagents or tools for measuring the concentration level of at least [Gal-3 and FABP4], and
c. means, reagents or tools for determining the AF type.

13. (canceled)

14. The In vitro method according to claim 2, wherein the method is carried out in persistent AF patients in which a catheter ablation is indicated for treatment.

15. The In vitro method according to claim 2, wherein the biological sample is a minimally-invasive biological sample.

16. The In vitro method according to claim 2, wherein the biological sample is plasma, blood or serum.

17. The In vitro method according to claim 2, wherein the patient has persistent AF in which a catheter ablation is indicated for treatment.

18. The In vitro method according to claim 17, wherein the biological sample is a minimally-invasive biological sample obtained from the patient.

19. The In vitro method according to claim 18, wherein the biological sample is plasma, blood or serum obtained from the patient.
